(19) United States
(12) Patent Application Publication
(10) Pub. No.: US 2017/0281190 A1
(43) Pub. Date: Oct. 5, 2017

(54) PRESSURIZED ANATOMICAL COMPRESSOR FOR KIDNEY DEVICE

(71) Applicant: Health Research, Inc., Buffalo, NY (US)

(72) Inventors: Khurshid GURU, East Amherst, NY (US); Lauren SAMAR, Buffalo, NY (US)

(21) Appl. No.: 15/513,235

(22) PCT Filed: Sep. 23, 2015
(86) PCT No.: PCT/US15/51801

§ 371 Date: Mar. 22, 2017

Related U.S. Application Data
(60) Provisional application No. 62/054,115, filed on Sep. 23, 2014.

(51) Int. Cl. A61B 17/12; A61B 17/135

(52) U.S. Cl. CPC A61B 17/12; A61B 17/135; A61B 2017/00022

(57) ABSTRACT

A surgical apparatus has connected strips that are configured to surround an organ, such as a kidney. The surgical apparatus at least partially mechanically occludes fluid flow into, out of or within part of the organ. Each strip is individually inflatable. Tubes deliver fluid to the strips. Each of the tubes is connected to one of the strips.

### CROSS-REFERENCE TO RELATED APPLICATIONS

This application claims priority to U.S. Provisional Application No. 62/054,115, filed on Sep. 23, 2014, now pending, the disclosure of which is incorporated herein by reference.

### FIELD OF THE INVENTION

This invention relates to a device used during surgery and, more particularly, to a device used during partial nephrectomy (PN) kidney surgery.

### BACKGROUND OF THE INVENTION

In the field of urology surgery, partial nephrectomy (PN) surgeries are performed to remove renal tumors while sparing as much of the remaining kidney as possible. As kidneys are highly vascular structures, receiving roughly 22% of the cardiac output with many branches of veins and arteries, operations on the kidneys are at risk for major blood loss without appropriate preventative methods.

In a typical open PN surgery, the surgeon directly accesses the kidney by way of an incision. The renal hilum (renal artery, renal vein, and urethra) is clamped and the whole organ is cooled to preserve the organ for as long as possible thereby reducing postoperative damage.

PN surgery may also be performed using minimally-invasive surgery techniques where the surgeon makes small incisions in the patient and the kidney is handled indirectly with long instruments inserted through the incisions. The instruments have ergonomically designed handles operable by the surgeon for controlling movement of distal ends that have surgical instruments (scissors, graspers, etc.) Among urologists, minimally-invasive PN surgery is gaining popularity due to the reduced risk for infection, less pain, and shortened hospital stays. A method employed in minimally-invasive procedures is to reduce blood flow to and from the kidney by clamping the renal hilum, which creates a mostly bloodless surgical field. Placing and retrieving ice slush inside the patient's body to cool the kidney is difficult and rarely attempted, so the time window to perform the entire surgery is shortened to avoid renal injury or eventual failure due to ischemia.

A range of studies has been performed to examine the relationships between clamping (fully or partially), ischemia time (cold or warm), tumor sizes, blood loss, acute renal failure, and renal injury. Some results indicate the longer the kidneys are without blood flow after a certain time (such as, for example, roughly 30 minutes), the higher the risk of postoperative renal injury or failure. If the patient has a solitary kidney, this time is shortened even further. There are other variables (age, gender, body mass index, etc.) that could affect the risk, but the general trend in PN studies is that the longer the kidney experiences ischemia, the greater the risk to patient outcome. Due to these trends, there is a need for a device and method to create a relatively bloodless surgical field without clamping the renal hilum to avoid the potential injury to or failure of the kidney caused by the clamping.

### BRIEF SUMMARY OF THE INVENTION

A pressurized anatomical compressor for kidney device (“PACK'D” or “device”) is disclosed. This device can be used during open, laparoscopic, robotic, or other types of PN kidney surgeries. The device can be used in surgery to remove the need to perform arterial occlusion via clamping of the renal artery and vein.

The device disclosed herein at least partially mechanically occludes fluid flow into, out of, or within a controlled part of an organ. The organ may be a kidney, though the device disclosed herein also could be used with other human or animal organs.

The device may be embodied as an adjustable cage that fits around an organ, such as a kidney. In general, the device has a cage with inflatable strips connected to a tubing system that is controlled by a valve system and a pump. The adjustable cage, which may be loose and not adjusted for a specific kidney, has several connecting strips that form a cavity for the kidney. Each strip may be configured as a long strip that passes over or around the external curve of the kidney with the ends of the strip on the internal curve of the kidney. The ends of a strip also may be on the external curve of the kidney. There may be strips connected to the strips near the ends of the kidney between the external and internal curves of the kidney and that wrap around those ends. These strips can be flexible, structured with one end for each side, or rigid.

The ends of the strips may be designed for adjustability and a non-slip fastener can hold the ends of each strip together. The mode of fastening is compatible to the standard minimally-invasive surgical devices as well as simple to fasten in open surgeries. The fastener may be a ring that encloses the ends and can slide along the ends for adjustability.

The strips can create at least two divided portions of the kidney or other organ. One portion has reduced blood flow or fluid flow compared to the other portion or portions. One portion may or may not be symmetrical to the other portion or portions. One portion may or may not be the same size as the other portion or portions.

The strips may be connected to each other at various locations of the organ, such as, for example, at the centerline of the kidney or other organ. For example, there may be one strip on each side of the centerline of the kidney. The strips can be perpendicular to the strips they are connecting. The strips may form obtuse angles on one side of the strips to be connected. The strips also may be above or below the standard centerline of the organ.

In an example, there may be two strips with one connector between the strips and another strip on the end perpendicular to each of the strips. In another example, there are four strips with three connecting strips and no strips at the ends. In yet another example, there are more or less than four strips. There also may be no strips at the ends or the device may be designed such that not all of the neighboring strips are connected to one another.

There may be no supplementary material between each strip. In other embodiments, there may be material, such as mesh, fabrics, or plastics, between some or all of the strips.

The device may be fabricated of biocompatible materials. For example, the device may be fabricated of silicone.

There may be inflatable components (i.e., bladders) embedded in or otherwise connected to each strip. The inflatable components can be connected to a tube system that carries air or other fluid for inflating the corresponding bladder. The inflatable components can expand into the cavity containing the organ. The outer surfaces of the strips (e.g., surfaces of the strips opposite of the kidney surface, not contacting the kidney surface, or not facing the kidney surface) may be rigid or semi-rigid to contain or direct the inflatable components. The inflatable components may be curved or have other cross-strips. In an example, inner inflatable components can be divided into smaller interconnected parts that each has a shaped face (such as square or rectangular) contacting the surface.

The inflatable components of the strips may be separated into two parts: one part that covers the external curve to the center of the strip on both sides and the other part covers the internal curve to the center of the strip on both sides. Each component may be defined as a strip and since the internal curve may contain the ends, there are two sub-strips in the internal curve component with one on each side of the kidney. The external curve component may have one connecting point with the tube system since it is continuous while the internal curve component may have two connectors since it is discontinuous. The fasteners around the ends on the internal curve strips can block unnecessary inflating of the strip (or sub-strip) or the part of the strip not in contact with the kidney.

In an example, there can be a tubing system having a tube in fluid communication with each inflatable strip and having a control system. There may be one tube for each inflatable strip along the internal and external curves and one tube branches into two for each sub-strip of the internal curve strips due to the discontinuity.

In another example, there is at least one small tube for each inflatable strip. The tube for each strip is collected into a bundle leading to the control system. The bundle may have a sheath which may enclose all of the tubes over a portion of the lengths of the tubes.

The tubes may collect at any location convenient to the requirements of the particular organ and/or surgery. For example, the tubes may collect into a bundle at an end of a center strip. The location where the tubes collect on the cage can be at the center of the center connecting strip, though the collection point on the cage can be at any intersection. There may be a location where the tubes are configured to collect on both sides of the organ and the tubes from either side collect into a bundle at a distance from the cage.

The location where the tubes collect may be at a distance from the cage sufficient for the branches to be moved to different sides of the organ depending on the preference of the surgeon or the desired field of view.

The control system may be a valve system with controls for selectably inflating the strips. The control system can have different shapes and manners of arrangement. In an example, a series of stopcocks form a manifold. The manifold may comprise a stopcock corresponding to each inflatable component of the device. In another example, the control system comprises a plurality of stopcocks arranged in a circular manner. In yet another example, a circular device with a valve system controls the fluid flow into and out of the device.

Some or all of the stopcocks can be 4-way stopcocks for total control of the air or fluid flow. 3-way, 2-way, or other stopcocks can be used. Each stopcock or valve can correspond to an inflatable component. Color-coded or other indications may show the corresponding valve or stopcock and inflatable component. The indications may be visual, tactile, or other types of indication and combinations of such types.

The manifold can be connected to a pump that inputs and removes the air or other fluid from the inflatable strips. This pump can be a handheld pump, a machine pump, or other type of pump.

### DESCRIPTION OF THE DRAWINGS

For a fuller understanding of the nature and objects of the invention, reference should be made to the following detailed description taken in conjunction with the accompanying drawings, in which:

FIG. 1 is a perspective view of a device on a kidney according to an embodiment of the disclosure;

FIGS. 2A-2B are perspective views of a device on a kidney showing various tube collection locations;

FIGS. 3A-3B are perspective views of an example of a system with the device and shaded strips illustrating corresponding tubes;

FIG. 4 is a perspective view of an example of an external component;

FIG. 5 is a perspective view of another example of an external component; and

FIG. 6 is a perspective of the device on a kidney during a procedure.

### DETAILED DESCRIPTION OF THE INVENTION

Although claimed subject matter will be described in terms of certain embodiments, other embodiments, including embodiments that do not provide all of the benefits and features set forth herein, are also within the scope of this invention. Various structural, logical, process step, and electronic changes may be made without departing from the scope of the invention.

Directional terms are used in the following description to indicate relative reference only, and should not impose any limitations on how any apparatus or components are to be manufactured or positioned during use. Here and throughout, for clarification and reference purpose only, the external curve of the kidney is the curve opposite of the renal hilum while the internal curve of the kidney includes the renal hilum.

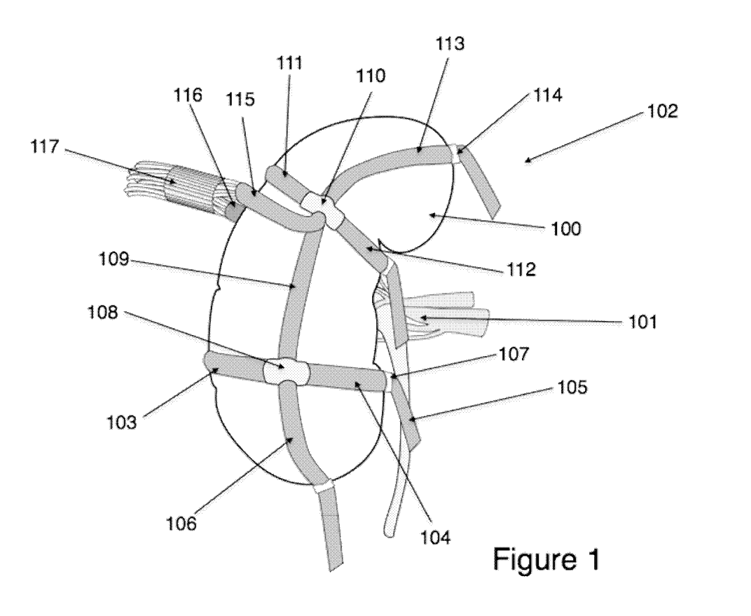

FIG. 1 is a perspective view of a device 102 on a kidney 100 according to an embodiment of the disclosure. The device 102 comprises a plurality of strips 103, 104, 106, 109, 111, 112, 113, each of which may be connected to one or more of the other strips. The connected strips of the device may be considered as forming a cage having a cavity configured to encompass an organ. For example, device 102 is illustrated with a kidney 100 within the cavity defined by the strips 103, 104, 106, 109, 111, 112, and 113. While only one side of the device 102 and the kidney 100 is shown in FIG. 1, the device 102 may have a similar arrangement on the opposite side of the kidney 100. The device 102 comprises a plurality of tubes 117 for delivering fluid to the strips. Each tube of the plurality of tubes 117 is in fluid communication with a corresponding strip.

The device 102 may be configured such that the renal hilum 101, comprising the renal artery, renal vein, and urethra, protrudes from the cavity of the device 102 when a kidney 100 is contained in the device 102. Two strips 103 and 104 are shown below the renal hilum 101 and connected to an intersection 108. On the external curve relative to the renal hilum 101, strip 103 is continuous from the intersection 108 to a corresponding intersection (not shown) on the opposite side of the kidney 100. On the internal curve relative to the renal hilum 101, strip 104 is discontinuous, separating into two sub-strips 105 from the intersection 108. This strip 104 contains two sub-strips 105, one on each side of the kidney 100, with the sub-strips 105 fastened together against the surface of the kidney 100 by a fastener 107. On the bottom half of the kidney 100, there is another strip 106 that accommodates the end curve of the kidney 100. Strip 106 is configured with two sub-strips (similar to strip 104) with the sub-strips fastened together by a fastener. Strip 106 may be connected to intersection 108 on either side of the kidney 100. At the opposite end of the kidney 100, a structure similar to that of strips 103, 104, and 106 connected by intersection 108 is present with strips 111, 112 and 113 connected by intersection 110. A fastener 114 fastens the sub-strips of strip 113 to one another.

Connecting these two sets of strips is a strip 109, connecting intersection 108 to intersection 110 on both sides of the kidney 100. This strip 109 may provide reinforcement to prevent the device 102 from undesired separation from the kidney 100. On either end of the strip 109, intersections 108 and 110 can control the position of the inflatable strips or the tubing. A tubing system (not illustrated in FIG. 1) is connected to a bundle of tubes 117 leading away from the device 102 and the kidney 100. Each tube may be directly or indirectly connected to a corresponding strip such that the strip is in fluid communication with the tube. For example, each tube may end at an intersection 108, 110. The tubes of the device 102 may not be arranged symmetrically on the two sides of intersections 108 and 110. The strips 103 and 111 can require one tube to feed the inflatable portion. The strips 104, 112 and strips 106, 113 can require two tubes, one for each side of the kidney 100. The strip 109 may be fed from only intersection 110 and intersection 108 can serve solely as an anchor at strip 109. Therefore, at intersection 110 there can be three tubes feeding into specific strips at intersection 108. On the opposite intersection of intersection 110, there can be two tubes feeding into the opposite side of strip 104 and strip 106. At intersection 110 on one side, there can be a total of four tubes feeding each of the connecting bands. On the opposite intersection 110, there can be three tubes feeding the opposite side of the strip 112, strip 113, and strip 109. Intersection 110 may serve as the collection point of all the tubes in the device 102. The intersection 110 and its opposite intersection exist on both sides of the device 102 and extends away from the kidney 100 in sub-bundles 115 and 116. The sub-bundles 115, 116 combine into one larger bundle of tubes 117. This bundle of tubes 117 leads to the external component of the device 102.

One or more strips may comprise an inflatable component, such as, for example, a bladder. In one example, the strip is a bladder. In another example, the inflatable component is positioned between the strip and the kidney surface. In another example, the inflatable component is positioned in the strip against the kidney surface. The strip may be rigid or semi-rigid to enable or direct expansion of the inflatable component or strip against the kidney surface. In another example, one or more strips includes the inflatable component and the strip itself inflates.

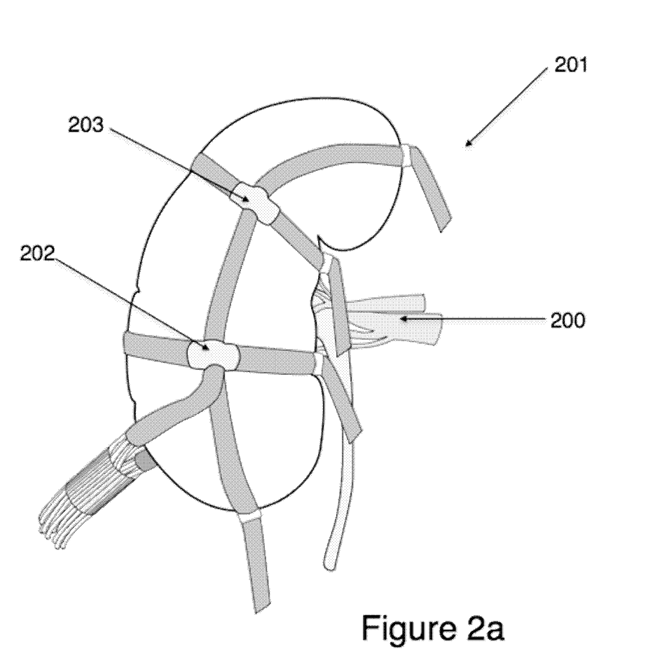

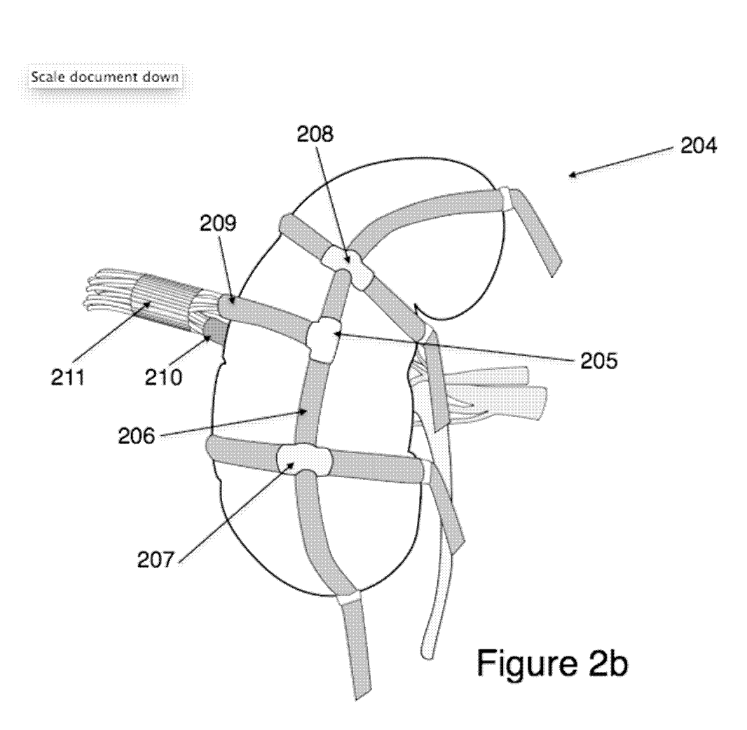

FIGS. 2A-2B are perspective views of a device 201 or device 204 on a kidney showing different tube collection points. In FIG. 2A, the structure of the strips are the same as FIG. 1, but another position for the collection point 202 is illustrated. Instead of a collection point at the intersection above the renal hilum 200, the collection point 202 is below the renal hilum 200. Intersection 202 of FIG. 2A may be the same as intersection 110 in FIG. 1 and intersection 108 in FIG. 1 may be the same as intersection 203 in FIG. 2A in terms of the amount of tubing feeding at the respective intersections and how the collection point of the tubing is arranged.

In FIG. 2B, the structure of the strips may be the same as presented in FIG. 1. The collection point 205, at the center of the middle strip 206, is presented in FIG. 2B. The middle strip 206 is anchored to intersections 207 and 208. The tubing system may be changed accordingly. For example, at intersections 207 and 208 on one side, there are three tubes at each intersection feeding into the corresponding strips. On the opposite side of the device, there are two tubes at each intersection. The strip 206 is fed air or other fluid at collection point 205 on either side of the device 204. The collection points lead outward to sub-bundles 209, 210 and combine into one bundle of tubes 211 that leads to the external component of the device 204.

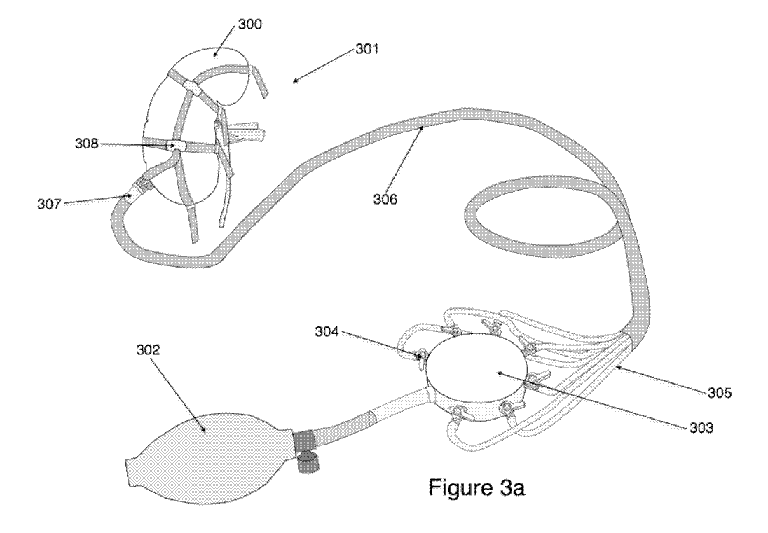

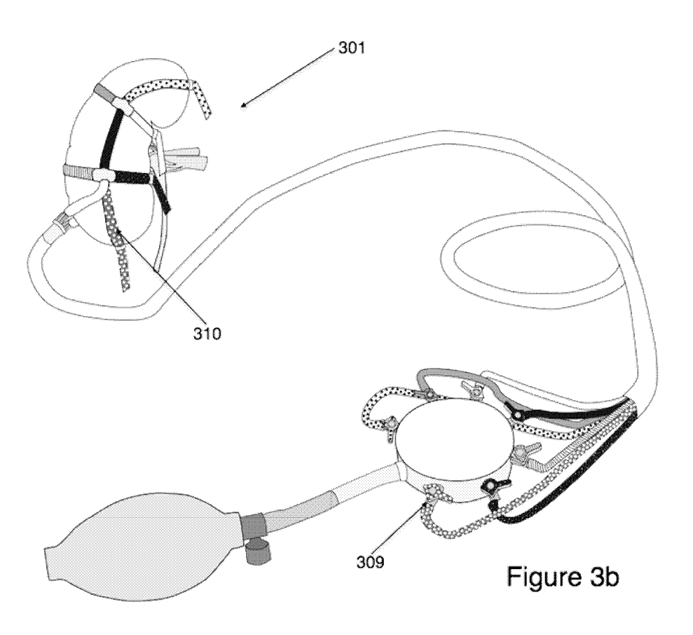

FIGS. 3A-3B are perspective views of an example of a system with the device 301 and shaded strips illustrating corresponding pipes. The device 301 on the kidney 300 may be similar to that illustrated in FIG. 1 or FIG. 2A. Strips to be inflated on the device 301 on the kidney 300 are selected by an operator (e.g., a surgeon) and each of the valves 304 on the control system 303 is adjusted to block or allow airflow. The handheld pump 302 pumps air into the control system 303. The air flows through the unblocked valves 304 and into the corresponding tubes 305. The tubes 305 are collected into a sheathed bundle 306 and the bundle 306 is divided at split point 307 into the sub-bundles connecting to either side of intersection 308 of the device 301.

In FIG. 3B, each valve 309 with shaded strips illustrates its corresponding strip 310.

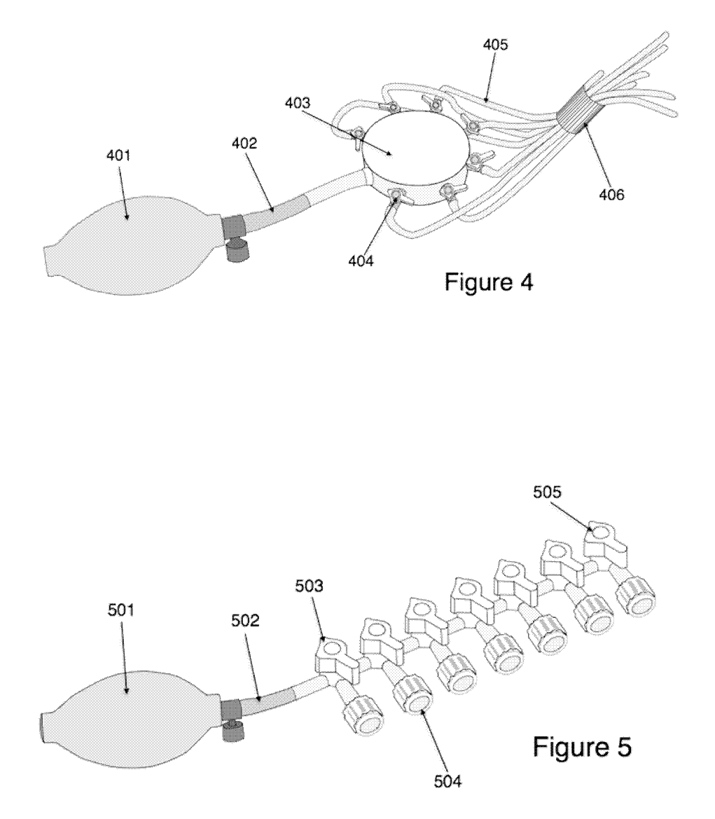

FIG. 4 is a perspective view of an example of an external component. There is a control system 403 connected to a hand-held pump 401 by a pump tube 402. The control system 403 comprises are seven valves switches 404 that allow an operator to control whether air flows into a corresponding tube 405. The position of the valve switches 404 can either allow or block flow of air or other fluid. Each valve switch 404 has a corresponding tube 405 and the tubes 405 are collected into a bundle 406 leading away from the control system 403. This bundle 406 can make insertion into the patient and operation easier during a surgery because fewer tubes 405 are loose or free to be moved proximate or inside the patient. Each tube 405 corresponds to one strip on the device.

FIG. 5 is a perspective view of another example of an external component. There is a series of seven stopcocks 503, 505 connected to a hand-held pump 501 by a pump tube 502 between the manifold and the pump 501. Each stopcock 503, 505 has a tube connector 504 extending from the manifold system. Each stopcock 503 with the exception of the last stopcock 505 in the series leading away from the pump 501 may be a 3-way stopcock. Turning the handle on the stopcocks 503, 505 controls the flow of air or other fluid.

For example, when the handle on the stopcock 503 is pointed toward the tube connector 504, fluid flows through the manifold and into the corresponding tube connector 504. When the handle on the stopcock 503 is pointed in the opposite direction of the tube connector 504, the fluid flows through the manifold at that strip without flowing into the tube connector 504. When the handle on the stopcock 503 is pointed toward the pump 501, the fluid flows from the manifold and into the respective tube connector 504 without flowing to the rest of the manifold system downstream of the stopcock 503 pointed toward the pump 501. When the handle on the stopcock 503 is pointed toward the end of the manifold opposite the pump 501, the fluid flow is blocked from the rest of the manifold and the respective tube connector 504.

The last stopcock 505 is a 2-way stopcock. When the handle on the stopcock 505 is pointed toward the tube connector 504, fluid flows from the manifold into the tube connector 504. When the handle on the stopcock 505 is pointed away from the tube connector 504, the fluid flow is blocked from the tube connector 504 and does not pass through the stopcock 505.

At each stopcock 503, 505, a tube is connected to the tube connector 504 and each tube corresponds to a strip of the PACK'D. Strips are selected to be inflated by adjusting each stopcock 503, 505 depending on the position on the manifold and the desired inflation of PACK'D.

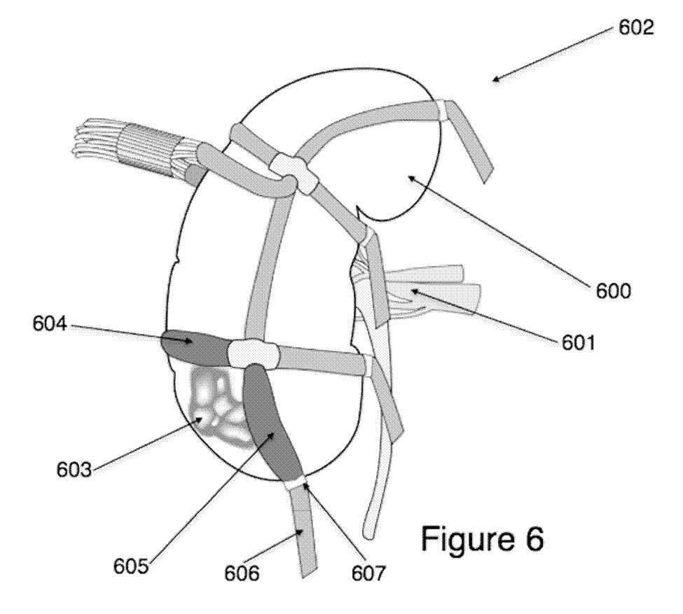

FIG. 6 is a perspective of the device 602 on a kidney 600 during a procedure. In FIG. 6, there is a tumor 603 on the kidney 600 and the appropriate strip 604 and strip 605 are inflated to restrict blood flow into that portion of the kidney 600. It should be noted that, in this embodiment, the ends of the sub-strips 606 of strip 605 are not inflated due to the ring fastener 607.

In the example of FIG. 6, the tumor 603 is on the external curve toward one end of the kidney 600. The patient may be prepared for a laparoscopic PN surgery. When all the necessary ports are placed in the patient, the surgeon can introduce the instruments to begin the surgical procedure and the device 602 is inserted into the body via cannula and opened to its loosened structure. The kidney 600 is inserted into the cavity of the device 602 with half of the strips of the device 602 on either side of the renal hilum 601 and the fasteners 607 on the internal curve relative to the renal hilum 601. The positioning of device 602 is adjusted based on where the tumor 603 is and the strips are tightened against the kidney 600 to secure the device 602. Because the tumor 603 is on the external curve toward the end of the kidney 600, the surgeon turns off all valves with the exception of those for the strip 604 and strip 605 next to the tumor 603. This prevents fluid flow to all strips of the device 602 except strip 604 and strip 605 surrounding the tumor 603. The surgeon pumps fluid into the strips 604, 605 with a pump until the strips reach an effective pressure, which can depend on the patient's blood pressure or other factors. The valves may be set so that fluid cannot flow into or out of strips 604, 605, thereby maintaining the fluid pressure within those strips. The surgeon may make a small incision to observe the blood flow in the blood-restricted portion of the kidney 600 with the tumor 603. After confirming the restriction of blood flow, the surgeon can proceed with performing the PN surgery. At the conclusion of the procedure, the surgeon will turn the valve or valves to let the fluid out of the inflated strips 604, 605 and loosen the fasteners 607 from the strips having such fasteners. The device 602 will be slipped off the kidney 600 and be removed from the body. Closing procedure will follow to conclude the operation.

While a particular arrangement of strips is described herein, more or fewer strips may be used. The strips may be arranged (e.g., interconnected) in other configurations. For example, additional strips may be provided to narrow the regions of the kidney where blood flow is restricted or occluded. In another example, the device has only two strips. This may be used with, for example, exophytic tumors.

In an embodiment, the part of the device inserted into the patient can be compacted into a cylinder-like volume or some other shape and be inserted via port in minimally invasive procedures.

In an embodiment, the inflatable strips or strip can include sensors for blood flow or pressure.

In an embodiment, the inflatable strips or connectors between the tubes connecting the inflatable strips may be airtight or otherwise fluid-tight.

In an embodiment, the fasteners may be clips, slip-type fasteners, or any other fastener.

The PACK'D may not completely block blood flow to the portion of the kidney or other organ being operated on.

The dimensions of the PACK'D can vary for different patients, organ sizes, or types of organs. The dimensions of the strips also can vary. In an example, the strips are approximately 4 cm to 6 cm in length.

The PACK'D reduces or eliminates the need for clamping the renal artery by restricting blood flow to certain parts of a kidney. The remainder of the kidney receives blood. This may reduce renal injuries during and after a surgery. This also may lengthen time available for a surgeon to operate on a kidney without damaging the kidney.

Although the present disclosure has been described with respect to one or more particular embodiments, it will be understood that other embodiments of the present disclosure may be made without departing from the spirit and scope of the present disclosure.

What is claimed is:

1. A surgical apparatus comprising:
a plurality of strips, each strip connected to one or more of the other strips, the strips configured to surround an organ, wherein at least one strip is inflatable; and
a plurality of tubes to deliver fluid to the inflatable strip(s), wherein each of the tubes is in fluid communication with a corresponding inflatable strip.

2. The surgical apparatus of claim 1, wherein the surgical apparatus is configured to at least partially mechanically occlude fluid flow into, out of, or within part of the organ.

3. The surgical apparatus of claim 1, wherein each of the strips comprises an inflatable component.

4. The surgical apparatus of claim 3, wherein each of the strips comprises an exterior portion opposite the organ that is at least semi-rigid.

5. The surgical apparatus of claim 1, wherein the strips are adjustable to fit around the organ.

6. The surgical apparatus of claim 1, wherein at least two of the strips are connected using a fastener to form a pair.

7. The surgical apparatus of claim 1, wherein each of the strips is fabricated of a biocompatible material.

8. The surgical apparatus of claim 1, wherein at least one of the strips comprises a sensor.

9. The surgical apparatus of claim 1, further comprising a pump connected to the tubes.

10. The surgical apparatus of claim 1, wherein the organ is a kidney.

11. A method comprising:
inserting a plurality of connected strips configured to surround an organ into a patient, wherein each of the strips is individually inflatable;
placing the organ into a cavity defined by the strips; and
selectively inflating at least one of the strips to at least partially mechanically occlude fluid flow into, out of, or within part of the organ.

12. The method of claim 11, further comprising tightening the strips around the organ prior to the selectively inflating.

13. The method of claim 11, wherein the strips are compacted prior to the inserting and are configured for a minimally invasive procedure.

14. The method of claim 11, further comprising:
deflating the strips;
slipping the strips off the organ; and
removing the strips from the patient.

15. The method of claim 11, wherein the organ is a kidney.
